(19) United States
(12) Patent Application Publication
(10) Pub. No.: US 2018/0146856 A1
(43) Pub. Date: May 31, 2018

(54) Multi-modality Dynamic Chemical Imaging of Fresh and Live Biological Tissue

(71) Applicant: Jaishankar Raman, Portland, OR (US)

(72) Inventor: Jaishankar Raman, Portland, OR (US)

(73) Assignee: iSpectrOm LLC, Portland, OR (US)

(21) Appl. No.: 15/364,608

(22) Filed: Nov. 30, 2016

(51) Int. Cl. A61B 5/00; A61B 5/08; A61B 5/02; G01N 29/24; G01N 21/35; G01N 21/65

(52) U.S. Cl. CPC A61B 5/0075; G01N 2291/02475; A61B 5/0082; A61B 5/4509; A61B 5/08; A61B 5/02; G01N 29/2418; G01N 21/35; G01N 21/65; A61B 2503/40; G01N 2021/3595; G01N 2201/06113; G01N 2201/08; G01N 2291/02466; A61B 5/0095

(57) ABSTRACT

This device and platform seeks to provide a point-of-care diagnostic tool to assess tissue quality. The handheld probe that interfaces with the tissue will have multiple components including fibers that deliver laser light, fibers that detect spectra, fibers that transmit acoustic signals and fibers that conduct force displacement data electronically. The light is generated from a laser light source within the detection console. This console also houses the spectroscopic detector arrays, IR, Raman & others. In addition, if force transduction and/or elasticity is being assessed, the detecting capability to quantify this is also housed in this box. Further components include the ability to provide computation and correlative diagnostic results. Some of these data may be stored in a web-based data storage capability with the ability to utilize large databases of histology images.

### FIELD OF INVENTION

A point of care technology that utilizes multi-modality imaging using spectroscopy and related technologies to assess and quantify the health and pathology of the underlying tissues.

### BACKGROUND OF THE INVENTION:

Tissue quality is difficult to quantify during endoscopy, interventional procedures and surgery. This is also difficult to assess effectively in the intact patient.

Pre-procedure imaging such x-rays, CT scans, PET scans, nuclear scans, ultrasound scans and MRI often provide good information but are not specific about tissue quality. They do not provide information about cellular characteristics, chemical composition, structure and handling capabilities for surgical procedures. Tissue fragility and frailty, which are important in older patients, radiation induced tissue injury, immunosuppressed individuals, are difficult to identify and quantify.

During invasive procedures such as endoscopy, angiography, surgery, proceduralists often assess tissue indirectly or by the “feel”, commenting on the quality of the tissue underneath. There is no way to know if the tissue is benign or malignant, inflamed or infected, ischemic or scarred, resectable or not, without confirmatory histopathological examination. Even then, this information is not available immediately because conventional histopathology involves removal of some tissue and a result available in 24 to 48 hours. Chemical details of live cells may also change during the preparation process for histopathology.

BRIEF SUMMARY OF THE INVENTION

Spectroscopic imaging is emerging as a powerful new tool for non-destructive chemical imaging in medicine. By detecting the wavelength of light scattered off a particular surface, and analyzing the resulting spectra, the chemical composition of the material can be revealed. The combination of different spectroscopic components allows detailed assessment and imaging of tissue.

This invention is a point of care technology that utilizes multi-modality imaging using spectroscopy and related technologies to assess and quantify the health and pathology of the underlying tissues.

### BRIEF DESCRIPTION OF THE DRAWINGS

FIG. 1 shows an overview of the system with the probe containing multiple fibers, connected by a fiber optic cable bundle to the console, which performs spectroscopy & other imaging. This console connects with a CPU that performs the analyses along with the computation resulting in automated tissue characterization or diagnosis.

FIG. 2 shows a magnified view of a simple tissue assessment probe with the different components of its fiber optic cable bundle and its sterile cover.

### DETAILED DESCRIPTION OF THE INVENTION

The proposed invention is a multi-modality probe and platform that combines multiple imaging modalities including photoacoustic imaging, spectroscopy (Infrared and Raman), and light diffraction capabilities—to give a composite view of tissue (live or dead) quality. In addition, this approach will also provide chemical imaging data on the scanned tissue that allows cellular and inter-cellular resolution. Additional high-resolution probe use can promote detailed cellular pathological imaging.

This provides information about
Tissue quality and stiffness
Chemical composition and content of chemicals such as protein, fat, fatty acids, sugars, specific metabolites, DNA methylation products, and others
Density of tissues
Strength of tissue & healing capability
Microscopic structure

This is the first objective quantification of tissue quality that can be performed as point of care or point of intervention application. Moreover, it is based on a portable and handheld device.

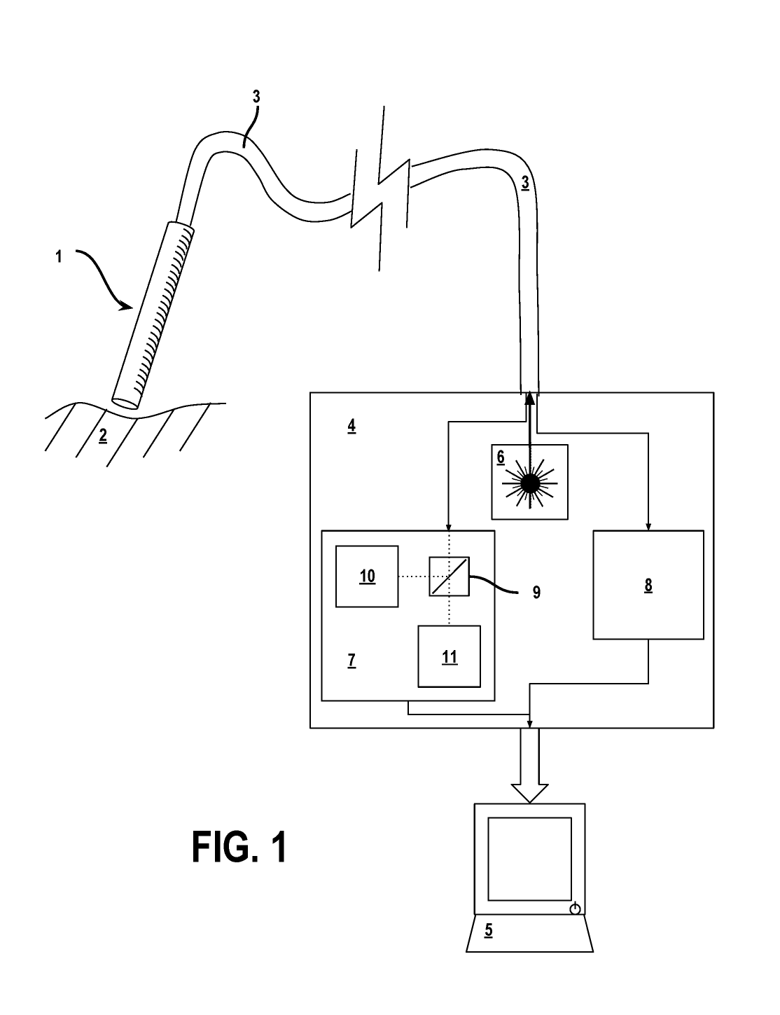

FIG. 1 shows a schematic diagram of the entire system. The hand-held imaging probe 1 is shown scanning an area of human or animal tissue 2. The probe 1 will be connected via a fiber optic bundle 3 to a console 4 and CPU 5 that provides a readout or an objective quantification of the state of the underlying tissue. The console 4 houses the tunable laser light source 6, the spectroscopy detector array 7, and detector systems for other modalities 8 (such as photoacoustic imaging or force sensing). The spectroscopy detector array has a beam splitter 9 and distinct Infrared 10 and Raman 11 spectroscopy arrays for simultaneous acquisition. Arrows in the diagram show the direction of data/information flow along optical fibers.

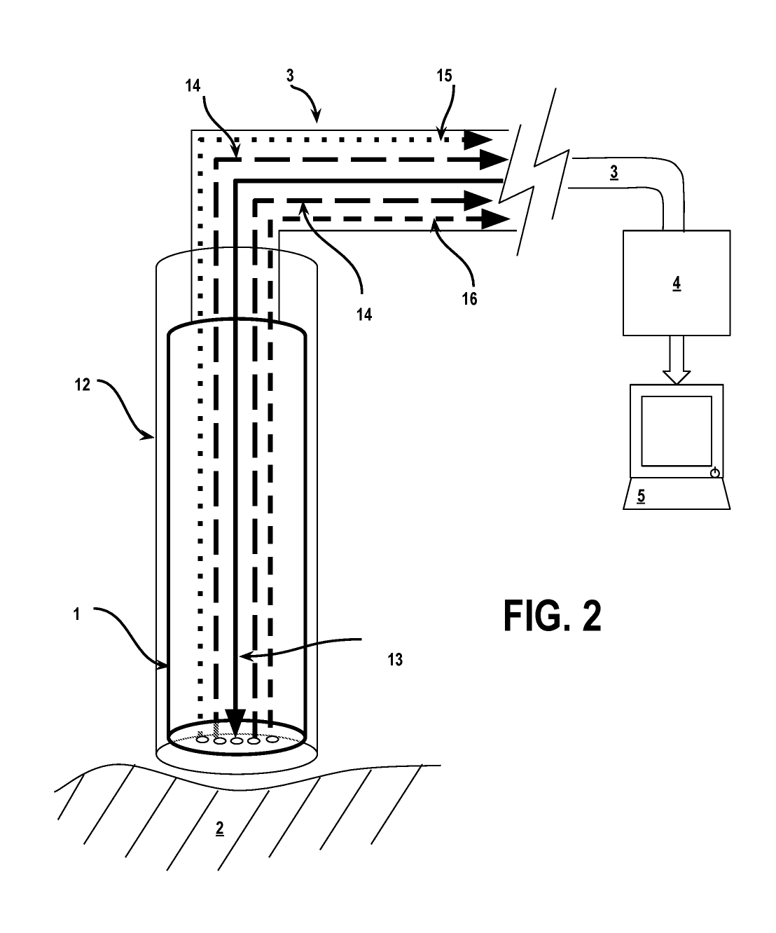

FIG. 2 shows a close-up cross-sectional view of the probe and its different components in relation to the system as a whole. A sterile sheath 12 covers the probe when in use in sterile/hospital settings. The fiber optic bundle 3 contains multiple optical fibers for both transmitting and receiving spectroscopic and other signals. The excitation fiber 13 delivers different wavelengths of laser light from the tunable laser light source 6 shown in FIG. 1 to the animal or tissue sample 2. The spectroscopic detection fibers 14 deliver scattered light of different wavelengths from the scanned tissue to the console 4 for specific detection in the spectroscopic detector array 7 shown in FIG. 1. Additional collection fibers for transmitting information collected from the probe to the console 4 are shown, a fiber for sending force transduction data 15 from a force transducer that could be housed in the probe, and a fiber for sending acoustic data 16 from acoustic detectors that could be incorporated into the probe. Analysis of collected data will be conducted in the console 4 and CPU 5. As in FIG. 1, arrows show the direction of data flow.

A simple low-resolution version of the device might just have a read-out suggesting normal, marginal/mild abnormalities or abnormal tissue character, in terms of integrity and healing potential (tissues undergoing surgical reconstruction). Another version may be geared towards assessing malignancy (suspicious areas for potential biopsy or excision). Alternate modes may be related to fibrosis and scarring (liver nodules, lung nodules, etc). Specific iterations in the heart may distinguish between normal myocardium, myocardium with ischemia and infarction, myocardium with hypertrophy and irreversible myopathic changes. High-resolution imaging can be activated by a mode switch, which uses predominantly FT-IR (Fourier Transform Infra-Red) and DFIR (Discrete Frequency Infrared) imaging, which along with computational methods, can be used to delineate the microscopic images of the tissue underneath. Raman Spectroscopy will also be used to provide a composite chemical composition based image along with the tissue map that has a depth penetration of around 2 to 3 cm. This again will have either a mode switch that allows complete scanning with FTIR & then Raman—till images are obtained, assessed and analyzed. This may be done sequentially either automatically or manually.

The probe and cable used to deliver laser light and detect signals would be standardized for a variety of laser energies and to detect an array of signals. The laser light source may be tunable or fixed depending on the fidelity of the device.

The spectroscopic diagnostic probe will have a laser light source and an array of detectors. The probe may also have a force transducer function or component that can assess tissue deformability or strength and acoustic detectors for photoacoustic and/or ultrasound imaging.

### EXAMPLES:

### Bone Quality Tester

We describe the Bone Quality Tester initially, as an example of a robust tissue quality assessment device. This will consist of a composite probe with multiple elements which can sense various parameters—calcium content, bone density, mineralization of bone, bone deformability, etc. These components are all incorporated into a handheld probe. The probe connects to a console that provides the readout. Individual aspects of the sensing capabilities will be able to provide more detail as required. For example, if a detailed calcium map of the bone surface is required, that may be possible. Assessment of areas of decreased calcification/weak areas of bone can be detected.

Applications:
All bone handling surgical specialties, including trauma and oncology.
Assessment of bone quality during operative intervention.
Likely quantification of osteoporosis, osteopenia, etc.
Assessment of bone quality during and after completion of reconstructive surgery.
Evaluation of bone viability in the presence of infection.
Bone margins after resection of localized malignant lesions.

### Tissue Quality Assessor in the Heart & Myocardium

This probe will ideally differentiate between normal, ischemic (decreased blood flow) heart muscle and scarred heart tissue. Additionally, the mode switch may allow more sophisticated diagnoses such as evaluating the extent of fibrosis in the muscle or the amount of hypertrophy of the cardiac muscle. This probe will aid surgeons intraoperatively in decision-making.

### Lung Tissue Scanner

This will assess the lung surface and tissue up to 2 cm deep during VATS (video-assisted thoracoscopic surgery). This will predominantly be used to assess and evaluate suspicious areas in the lung and the mediastinum, prior to biopsy or to guide biopsies.

### Dynamic Chemical Imaging & Microscopy

The combination of laser excitation and detection will be used to delineate a map of the underlying tissue using the biochemical signatures. The underlying cellular architecture can be accurately delineated and eventually a 3-dimensional reconstruction of the underlying tissue created.

Types of Tissue that can be analyzed:
Fresh tissue—fresh excisional or needle biopsy
Preserved tissue—tissue that is taken out by biopsy and preserved using formaldehyde
Live Tissue—In vivo imaging of live tissue without the need for excisional biopsy

1. A device platform that facilitates composite and complementary tissue imaging using multiple spectroscopic components and related sensory technologies, comprising:

2. A hand-held probe that interfaces directly with live tissue

3. A console that houses tunable laser light sources and arrays of detectors

4. A fiber optic cable bundle that conveys light and detected signals between said probe and said console

5. A CPU capable of analyzing and collating information from different detector sources and returning a diagnostic

6. wherein said imaging modalities in claim 1 include but are not limited to Raman spectroscopy, infrared spectroscopy, near infrared spectroscopy, Fourier transform spectroscopy, photoacoustic tomography, and others

7. wherein the related sensory technologies of claim 1 include but are not limited to ultrasound imaging, force sensing, and others

8. wherein the detector system of claim 3 has the capability to simultaneously analyze Infrared, Raman and other spectroscopies

9. A method for analyzing the convergence and collation of diverse light based, spectroscopic imaging techniques to provide an accurate chemical and histologic map of the adjacent tissue.

10. wherein the CPU of claim 5 has the capability to translate spectroscopic and other data to aid in creation of stainless histological maps

11. A method of automated analysis of collected data to allow detailed cellular, biochemical, metabolic, and structural data to be synthesized into actionable and meaningful diagnoses.

12. wherein the CPU of claim 5 allows for automated analysis of incoming data

13. wherein said CPU analysis results in specific characterization of tissue components

14. wherein said analysis of information is relayed in a rapid fashion

15. wherein said analysis returns actionable diagnostic information to the user
